(19) United States
(12) Patent Application Publication
(10) Pub. No.: US 2025/0032129 A1
(43) Pub. Date: Jan. 30, 2025

(54) CANNULATED SCREW REMOVAL DEVICE AND METHODS

(71) Applicant: Acumed LLC, Hillsboro, OR (US)

(72) Inventor: Joseph Allan Pepin, Sachse, TX (US)

(21) Appl. No.: 18/767,658

(22) Filed: Jul. 9, 2024

Related U.S. Application Data
(60) Provisional application No. 63/528,955, filed on Jul. 26, 2023.

(51) Int. Cl. A61B 17/16; A61B 17/88

(52) U.S. Cl. CPC A61B 17/1631; A61B 17/888

(57) ABSTRACT

Provided herein are a cannulated screw removal device and methods for removing a cannulated screw from bone using a cannulated screw removal device. The cannulated screw removal device may include an elongated shaft and a collar configured to receive the elongated shaft. The elongated shaft may include one or more threads configured to secure the elongated shaft to a cannula of a cannulated screw. The collar may include one or more protrusions configured to engage with at least a portion of the cannulated screw so that the collar can deliver a torque to the cannulated screw.

### PRIORITY CLAIM

This application claims priority to U.S. Provisional Patent Application No. 63/528,955 filed on Jul. 26, 2023, the entire contents of which are hereby incorporated by reference and relied upon.

### BACKGROUND

Bone fixation devices, such as bone screws, are frequently used for the attachment of implants to bone, bone to bone fixation, or for soft tissue fixation. Due to various causes, implanted screws may break during implantation or after implantation into the bone. If the reason for implanting the screw has not resolved or if the broken screw is further damaging the bone, the broken screw should ideally be removed and replaced with a new screw. Surgeons may be able to easily remove the first screw fragment (with a head) using a driver. However, the challenge or problem is that in some cases, the head of the screw may become so damaged that using a driver is no longer feasible. Additionally, the headless portion of the broken screw fragment embedded in the bone may be difficult or impossible to retrieve.

One way to remove broken or damaged screws embedded into the bone includes the use of a trephine that acts as a hole saw. The trephine fits over the outside of the embedded screw and cuts the embedded screw out of the bone. Another technique is to use a removal tip to grasp the outside threads of the embedded screw and apply a reverse torque to remove the fragment. However, the use of the trephine and the screw removal tip requires drilling a larger hole than desired (e.g., larger than the shaft/head of the screw) and removal of large amounts of bone.

Surgeons may use an easy out tool, which is a tapered reverse threaded tip on a hardened steel shaft. Axial force can be applied to the easy out tool while rotating the tool counterclockwise. However, the easy out tool tends to break due to strength limitations and also requires the use of manual axial force to engage with the screw. Surgeons often have difficulty removing broken screws from the bone while still retaining enough bone to replace the broken screw with one of similar size.

None of the current solutions solve the problem of removing the screw or screw fragment easily and reliably while still retaining a large enough amount of bone to reinsert another screw that is not substantially larger.

### SUMMARY

The present disclosure provides a new and innovative device and methods for removal of at least a portion of a screw from the bone, such as a cannulated screw. The cannulated screw removal device allows removal of cannulated screws, such as broken cannulated screws, without compromising too much bone and allowing the reinsertion of the next larger size screw. In some examples, a cannulated screw removal device is provided. The cannulated screw removal device may include an elongated shaft and a collar configured to receive the elongated shaft. The elongated shaft may include one or more threads configured to secure the elongated shaft to a cannula of a cannulated screw. The collar may include one or more protrusions configured to engage with at least a portion of the cannulated screw so that the collar can deliver a torque to the cannulated screw to remove the screw or screw fragment.

In some examples, a method of removing a cannulated screw using the cannulated screw removal device is provided. The method may include applying a torque to the elongated shaft in a first direction which causes the elongated shaft to screw into a cannula of the cannulated screw, securing the elongated shaft to the cannulated screw. The method may further include continuing to apply a torque to the elongated shaft until the collar engages with at least a portion of the cannulated screw; and applying a torque to the collar in the first direction to remove the cannulated screw from the bone.

Additional features and advantages of the disclosed methods are described in, and will be apparent from, the following Detailed Description and the Figures.

### BRIEF DESCRIPTION OF THE FIGURES

FIG. 1 illustrates an example cannulated screw removal device according to the present disclosure.

FIG. 2 illustrates a cross-sectional view of an assembled cannulated screw removal device according to an example of the present disclosure.

FIGS. 3A and 3B illustrate examples of an elongated shaft according to the present disclosure.

FIGS. 4A-4D illustrate a collar according to an example of the present disclosure. Specifically, FIG. 4A illustrates a perspective view of the collar; FIG. 4B illustrates a side view of the distal end portion of the collar; FIG. 4C illustrates a front view of the collar; and FIG. 4D illustrates a perspective view from the proximal end of the collar.

FIGS. 5-9 are diagrams illustrating a method of removing a cannulated screw form a bone according to an example of the present disclosure.

### DETAILED DESCRIPTION OF EXAMPLES

The present disclosure is directed to a cannulated screw removal device and a method for removing a cannulated screw from a bone using the cannulated screw removal device.

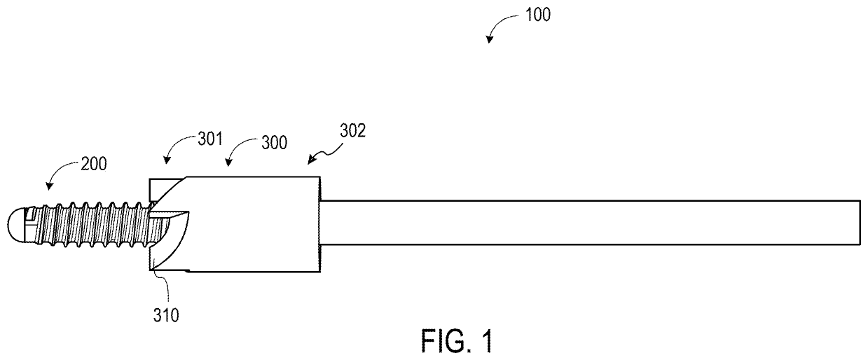

FIG. 1 shows a cannulated screw removal device according to an example of the present disclosure. The cannulated screw removal device 100 may include an elongated shaft 200 and a collar 300. In some examples, the collar 300 may include a cannula extending from a distal end of the collar 300 to a proximal end of the collar 300. The cannula of the collar 300 may be configured to receive the elongated shaft 200 so that the cannulated screw removal device 100 is in an assembled configuration as shown in FIG. 1. Stated differently, the collar 300 may be configured to surround at least a portion of the elongated shaft 200. When the elongated shaft 200 is received in the collar 300, the elongated shaft 200 and the collar 300 may rotate freely about one another along the same longitudinal axis. Alternatively, in some examples, the elongated shaft 200 and the collar 300 may be integrally formed.

The elongated shaft 200 may be configured to be inserted into a cannula of the cannulated screw for example, to facilitate the alignment of the cannulated screw removal device 100 with the cannulated screw. The collar 300 may be configured to engage with and mount onto a portion of the cannulated screw, such as an exposed surface of the cannulated screw. In some examples, the collar 300 may include one or more features, such as protrusions 310, on the distal end portion 301 of the collar 300 to engage with and secure to the cannulated screw.

The elongated shaft 200 when received within the collar 300 may act to align and center the collar 300 for mounting. The elongated shaft 200 may also transmit an axial force on the collar 300 to the cannulated screw, affixing or clamping the collar 300 to the screw, with a greater axial force than could be transmitted manually. Once the collar 300 is mounted onto the cannulated screw surface, a reverse torque may be applied to the collar 300 and to a portion of the cannulated screw to remove the screw from the bone.

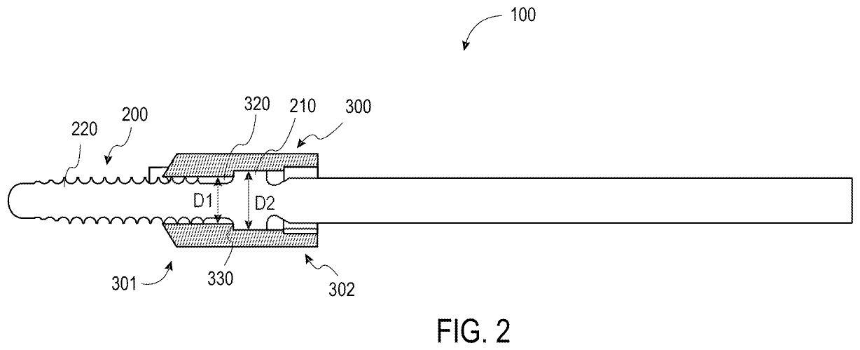

FIG. 2 shows a cross sectional view of the cannulated screw removal device 100 in an assembled configuration. As can be seen in FIG. 2, the elongated shaft 200 may include a stop head 210 and a cannulated screw engagement shaft 220. The diameter of the stop head 210 may be greater than the diameter of the cannulated screw engagement shaft 220.

In some examples, the cannula 320 of the collar may include a first diameter D1 and a second diameter D2. The first diameter D1 may be larger than the diameter of the cannulated screw engagement shaft 220 and smaller than the diameter of the stop head 210. The second diameter D2 may be larger than the diameter of the stop head 210.

The region between the portion of the cannula 320 with the first diameter D1 and the portion of the cannula 320 with the second diameter D2 may include a step portion 330. When the elongated shaft 200 is received in the cannula 320 of the collar 300, the stop head 210 may abut the step portion 330, preventing axial movement of the elongated shaft 200 within the collar 300 in one direction. The step portion 330 may also be used to apply an axial force from the stop head 210 to the collar 320, which may be used to mount the collar 320 onto the exposed surface of the cannulated screw.

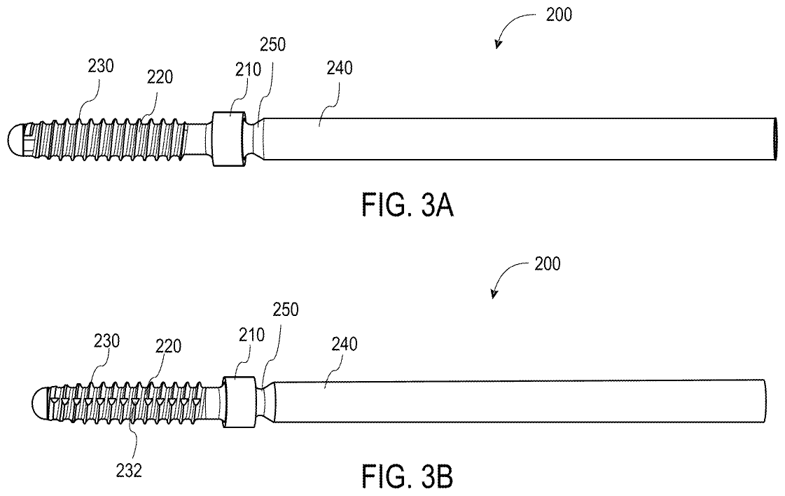

FIG. 3A shows an example of an elongated shaft 200 according to an example of the present invention. The elongated shaft 200 may include one or more threads 230 for engaging the elongated shaft 200 with the cannula of the cannulated screw. In some examples, the elongated shaft 200 may include one thread, two threads, three threads, four threads, or any number of threads.

The one or more threads 230 may be positioned on the cannulated screw engagement shaft 220. In some examples, the one or more threads 230 may extend along the whole length of the cannulated screw engagement shaft 220. In other examples, the one or more threads 230 may extend along a portion of the cannulated screw engagement shaft 220. In this case, the cannulated screw engagement shaft 220 may include a non-threaded portion (e.g., at the distal portion and/or proximal portion thereof).

The thread handedness of the elongated shaft 200 may be opposite from the thread handedness of the cannulated screw. For example, if the threads of the cannulated screw are “right-handed”, meaning the screw advances when rotating the screw to the right, then the one or more threads 230 of the elongated shaft 200 may be “left-handed.” This may allow the elongated shaft 200 to secure to the internal diameter of the cannulated screw and prevent removal of the elongated shaft 200 from the cannula of the cannulated screw when removing the cannulated screw from the bone.

The minor diameter of the cannulated engagement shaft 220 (e.g., the diameter of the cannulated engagement shaft 220 not including the threads 230) may be smaller than the diameter of the cannula of the cannulated screw. The major diameter of the cannulated engagement shaft 220 (e.g., the diameter of the cannulated engagement shaft 220 including the diameter of the threads 300) may be slightly larger than the diameter of the cannula of the cannulated screw. In some examples, the major diameter of the cannulated engagement shaft may be about 101% to about 120% of the diameter of the cannula of the cannulated screw. The larger diameter of the cannulated engagement shaft 220 with the threads 230 may allow the threads 230 to engage with and bite into the inner diameter of the screw, securing the elongated shaft 200 to the cannulated screw.

As shown in FIG. 3B, the elongated shaft 200 may include one or more cutting grooves 232, such as one cutting groove, two cutting grooves, three cutting grooves, four cutting grooves, or any suitable number of cutting grooves. The cutting grooves may extend along the full length of the one or more threads 230 or a portion of the one or more threads 230. The cutting grooves may provide better engagement of the cannulated screw engagement shaft 220 with the cannula of the cannulated screw.

Cannulated screws may have differing internal diameters. In some examples, the cannulated engagement shaft 220 may be custom made based on the internal diameter of the cannulated screw. In some examples, the elongated shaft 220 may be constructed so that the minor diameter is one thousandth, two thousandths, three thousandths, four thousandths, etc. of a millimeter smaller than the inner diameter of the cannulated screw.

In some examples, the major diameter of the cannulated engagement shaft 220 may be in a range of about 1 mm to about 5 mm. In some examples, the minor diameter of the cannulated engagement shaft 220 may be in a range of about 0.5 mm to about 4.5 mm.

In some examples, the threads 230 and/or the cannulated screw engagement shaft 220 may have a uniform diameter. In some examples, the threads 230 may include a taper as can be seen in FIG. 2. In some examples, at least a portion of the cannulated screw engagement shaft 220 (e.g., the shaft body itself without the threads) may include a taper.

In some examples, the distal end portion of the cannulated screw engagement shaft 220 may be flat. In some examples, the cannulated screw engagement shaft 220 may taper to a point or a tip at the distal end portion. When damaged, broken screws may become deformed, causing the cannula of the screw to no longer be uniform. Tapering of the cannulated screw engagement shaft 220 and/or the threads 230 may allow the elongated shaft 200 to fit into possibly deformed cannulas to allow lead in of the elongated shaft 200 while still providing enough engagement of the elongated shaft 200 with the cannulated screw to secure the elongated shaft 200 into the cannula of the screw.

In some examples, the distal end portion of the cannulated screw engagement shaft 220 may be rounded. For example, as shown in FIGS. 3A and 3B, the distal end portion of the cannulated screw engagement shaft 220 may be in the shape of a half sphere. Alternatively, the distal end portion of the cannulated screw engagement shaft 220 may include a rounded tip or be in the form of any rounded shape. The rounded distal end of the cannulated screw engagement 220 shaft may aid the cannulated screw engagement shaft 220 in finding the cannula of the cannulated screw and centering the cannulated screw engagement shaft 220.

Still referring to FIGS. 3A and 3B, the elongated shaft 200 may also include a torque delivery shaft 240. The torque delivery shaft 240 may be configured to deliver a torque to the cannulated screw engagement shaft 220 and/or the stop head 210 to advance the cannulated screw engagement shaft 220 into the cannula of the cannulated screw. Further, the torque delivery shaft 240 may allow the elongated shaft 200, such as the stop head 210, to transmit more axial force from the collar 300 to the cannulated screw than may be possible without the torque delivery shaft 240. For example, as the elongated shaft 200 is advanced into the cannulated screw, the stop head 210 may apply an axial force to the step portion 330 of the collar 300, traversing the collar 300 closer to the proximal end of cannulated screw surface. As the collar 300 contacts the cannulated screw surface, a torque may be continuously applied to the torque engagement shaft 240, further driving the stop head 210 into the step portion 330, which drives the protrusions 310 of the collar 300 into the cannulated screw.

In some examples, the torque delivery shaft 240 may be formed integrally with the stop head 210 and/or the cannulated screw engagement shaft 220. The torque delivery shaft 240 may be directly or indirectly connected to the stop head 210. In some examples, the torque delivery shaft 240 may be removably coupled to the stop head 210. For example, the torque delivery shaft 240 may include one or more features that couple with or engage with one or more features of the stop head 210. In some examples, the torque delivery shaft 240 may be a screw driver or some other kind of driving mechanism. In some examples, when the collar 300 and the elongated shaft 200 are assembled, the torque delivery shaft 240 may extend through the cannula of the collar 300 past the proximal end portion 302 of the collar 300. This gives a user enough room to apply a torque to the elongated shaft.

In some examples, the elongated shaft 200 may include a neck portion 250 between the stop head 210 and the torque delivery shaft 240. The neck portion 250 may have a lower torsional strength than the cannulated screw engagement shaft 220, the stop head 240, and the torque delivery shaft 240. Therefore, the neck portion 250 may experience torsional failure at a lower torque than the other portions of the elongated shaft 200. In some examples, the neck portion 250 may have a smaller diameter than the diameters of the cannulated screw engagement shaft 230, the torque deliver shaft 240, and the stop head 210. The neck portion 250 may taper from the diameter of the stop head 210 and/or the torque delivery shaft 240 to the smaller diameter as shown in FIG. 3. In some examples, the neck portion 250 may include an abrupt change in diameter from the stop head 210 and/or the torque delivery shaft 240. In some examples, the neck portion 250 may include one or more cutouts configured to lower the torsional strength of the neck portion 250 compared to the rest of the elongated shaft 200.

The lower torsional strength of the neck portion 250 is configured so that the torque delivery shaft 240 may break off from the stop head 210 and/or the cannulated screw engagement shaft 220 at a desired location, at a desired amount of torque, and/or in a desired way. The torsional strength of the neck portion 250 may be a torsional strength to ensure that enough axial force has been applied to the collar 300 so that it is sufficiently mounted to the cannulated screw. Additionally, the torsional strength of the neck portion 250 portion may ensure that excessive torsional strength is not applied to the device 100 and/or the screw. In this way, the neck portion may serve as a torque limiting feature. In some examples, the torsional strength of the neck portion may be in a range of about 0.05 Newton-meters to about 5 Newton-meters. The neck portion 250 may allow a surgeon to apply a consistent torque to the elongated shaft thereby applying a consistent axial force to the collar and may remove the necessity of guess work by the surgeon (e.g., in terms of how much torque the surgeon needs to apply to mount the collar 300 on the screw).

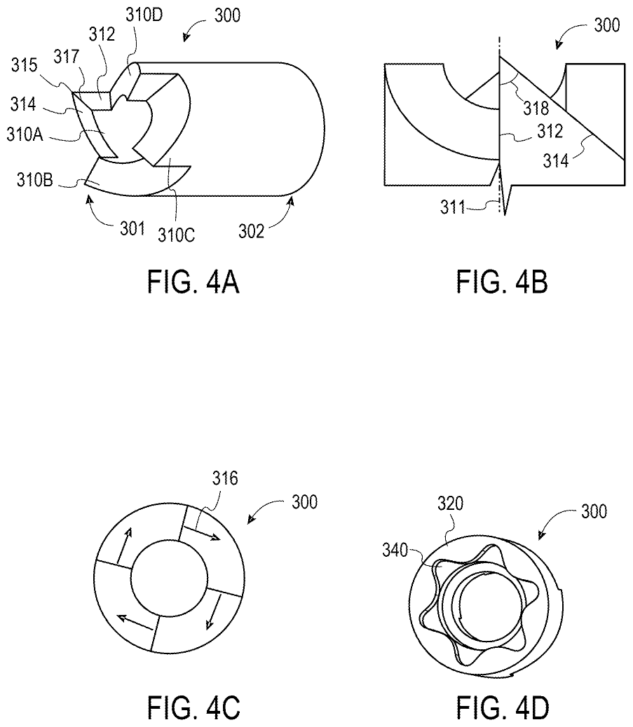

Referring to FIGS. 4A through 4D, the collar 300 may include one or more features, such as one or more protrusions 310A-D, used to seat the collar 300 into a portion of the cannulated screw. Although four protrusions are illustrated in FIG. 4A, the collar 300 may include more or less protrusions (e.g., 1, 2, 3, 5, 6, 7, 8, . . . ).

The protrusions 310A-D may extend from the distal end portion 301 of the collar. In some examples, the tip 315 may be sharply pointed and/or may form a sharp edge 317 such that the tip 315 or sharp edge 317 may bite into and deform a surface of the cannulated screw. In some examples, the sharp edge 317 may be substantially perpendicular to one or more virtual lines that are parallel to the central axis of the collar 300. In some examples, the sharp edge 317 may extend in a direction towards the center portion of the collar 300.

In some examples, the one or more protrusions 310A-D may include a steep slope portion 312. In some examples, the steep slope portion 312 may extend from the sharp edge 317. In some examples, the steep slope portion 312 may be substantially parallel to the central axis of the collar 300. In some examples, an angle formed between the steep slope portion 312 and the one or more virtual lines (e.g., 311 in FIG. 4B) that are parallel to the central axis of the collar 300 may be in a range of about 0° to about ±20°.

In some examples, the steep slope portion 312 may be angled in a direction from the one or more virtual lines 311 that are parallel to the central axis of the collar 300 to facilitate the application of a torque in a specific direction. For example, the steep slope portion 312 may be angled counterclockwise from the one or more virtual lines 311 that are parallel to the central axis of the collar 300 so that applying a counterclockwise torque to the collar 300 may result in a counterclockwise torque to the cannulated screw.

In some examples, the steep slope portions 312 of the one or more protrusions 310A-D may face a clock-wise direction 316 (when viewed from the distal end portion 301 of the collar 300) as shown in FIG. 4C.

In some examples, the one or more protrusions 310A-D may include a gentle slope portion 314. In some examples, the gentle slope portion 314 may extend from the sharp edge 317. The gentle slope portion 314 may be disposed between two adjacent steep slope portions 312. In some examples, an angle 318 formed between the gentle slope portion 314 and the one or more virtual lines (e.g., 311 in FIG. 4B) that are parallel to the central axis of the collar 300 may be in a range of about 30° to about 85°.

In some examples, the gentle slope portion 314 may be flat. In other examples, the gentle slope portion 314 may be curved, or include both a flat portion and a curved portion.

Once embedded in the cannulated screw, the protrusions 310A-D may be configured to provide a torque to the cannulated screw in a direction opposite from the orientation of the cannulated screw. For example, if the cannulated screw includes “right handed” threads, then to remove the screw from the bone, a torque may need be applied to the left or counterclockwise. In some examples, the tip 315/sharp edge 317 of the protrusion 310A-D may be disposed on the left most side of the protrusion 310A-D (when viewed from the outside of the collar 300 as shown in FIG. 4B) to allow application of a counterclockwise torque.

In other examples, if the cannulated screw includes “left handed” threads, then to remove the screw from the bone, a torque may need be applied to the right or clockwise. In this case, the tip 315/sharp edge 317 of the protrusion 310A-D may be disposed on the right most side of the protrusion 310A-D (when viewed from the outside of the collar 300) to allow application of a clockwise torque. In some examples, an angle formed between the steep slope portion 312 and the one or more virtual lines (e.g., 311 in FIG. 4B) that are parallel to the central axis of the collar 300 may be in a range of about 0° to about ±20°. In some examples, the steep slope portion is angled clockwise from the one or more virtual lines that are parallel to the central axis of the collar so that applying a torque to the collar may result in a clockwise torque to the cannulated screw. In some examples, the steep slope portions 312 of the one or more protrusions 310A-D may face a counterclockwise direction (when viewed from the distal end portion 301 of the collar 300). In some examples, an angle formed between the gentle slope portion 314 and the one or more virtual lines (e.g., 311 in FIG. 4B) that are parallel to the central axis of the collar 300 may be in a range of about −30° to about −85°.

In some examples, the protrusion 310A-D and/or the collar 300 may be made of a material having a hardness greater than the hardness of the cannulated screw. In some examples, the protrusion and/or collar may have a hardness in a range of about 36 HRC and about 60 HRC. In some examples, the distal end portion 301 of the collar 300 may include an internal, inverse taper, such as a substantially reverse conical shape. In some examples, the distal end portion 301 of the collar 300 may be flat or have any desired shape.

As shown in FIG. 4D, in some examples, the collar 300 may include a recess 340 on the proximal end of the collar 300. The recess 340 may be configured to engage with a driver. For example, the recess 340 may be shaped complimentarily to the shape of a driver. In some examples, the recess 340 may have a shape of a hexalobe, a cruciform, a square, a hex, a trilobe, or any other suitable shape. The driver may be used to apply a torque to the collar 300, which applies a torque to the cannulated screw. In other examples, a torque may be applied to the collar 300 without the use of a driver, for example, by directly rotating the collar 300 itself. In this case, the collar 300 may be elongated (and may have a length similar to the torque delivery shaft 240).

In some examples, the collar 300 may have a diameter substantially equal to or smaller than the outer diameter of the cannulated screw. This may allow the cannulated screw removal tool 100 to be used without the need for drilling a larger hole than necessary to remove the screw. In other examples, the diameter of the collar 300 may be larger than the outer diameter of the cannulated screw.

In some examples, the cannulated screw removal device 100 may be made out of (biocompatible) titanium, cobalt, stainless steel, and/or any other suitable biocompatible material. In some examples, the elongated shaft 200 and the collar 300 may be made out of the same material. In some examples, the elongated shaft 200 and collar 300 may be made out of different materials. In some examples, the cannulated screw removal device 100 may be a single-use disposable. In other examples, the cannulated screw removal device 100 may be for multiple use.

In some examples, a method of removing a cannulated screw using a cannulated screw removal device 100 according to an example of the present disclosure may be provided. The method may remove a cannulated screw, such as a cannulated screw fragment from a bone, for example, without removing large amounts of bone. The steps presented herein may be performed in any suitable order and combination, and may be modified by or combined with any of the other procedures and features disclosed herein.

In some examples, the method may include removing a first screw fragment from the bone. For example, if a screw breaks into two pieces while inside the bone, a surgeon may remove the first, proximal screw fragment (e.g., screw head portion). If the first screw fragment includes a head, the first screw fragment may be removed using a driver. Additionally or alternatively, the first screw fragment may be removed using the cannulated screw removal device 100 as will be described herein.

In some examples, the method for removing a cannulated screw may include removing tissue or portions of the bone so that there is enough clearance for the cannulated screw removal device 100 to reach the cannulated screw or cannulated screw fragment in the bone. In some examples, the method may include using a device, such as a reamer or a drill bit, sized similarly to the collar 300 of the cannulated screw removal device 100 to create a channel in the bone to fit the cannulated screw removal device 100.

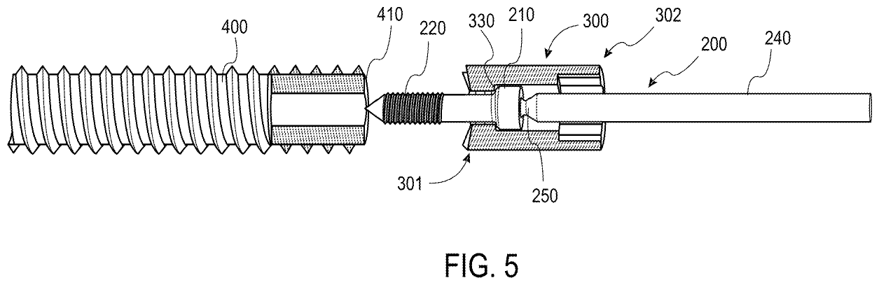

Referring to FIG. 5, the method may include assembling the cannulated screw removal device 100 by inserting the elongated shaft 200 into the cannula of the collar 300. The method may include inserting the distal end of the elongated shaft 200, such as the cannulated screw engagement shaft 220, into the cannula of the cannulated screw 400.

After insertion of the elongated shaft 200, the method may further include applying a torque to the elongated shaft 200, such as to the torque delivery shaft 240, in a first direction to screw the elongated shaft 200 into the cannula of the cannulated screw 400. Screwing the elongated shaft 200 into the cannula of the cannulated screw 400 may help secure the cannulated screw removal device 100 to the screw 400. Additionally, it may help align and center the collar 300 of the screw removal device 100 with the cannulated screw 400.

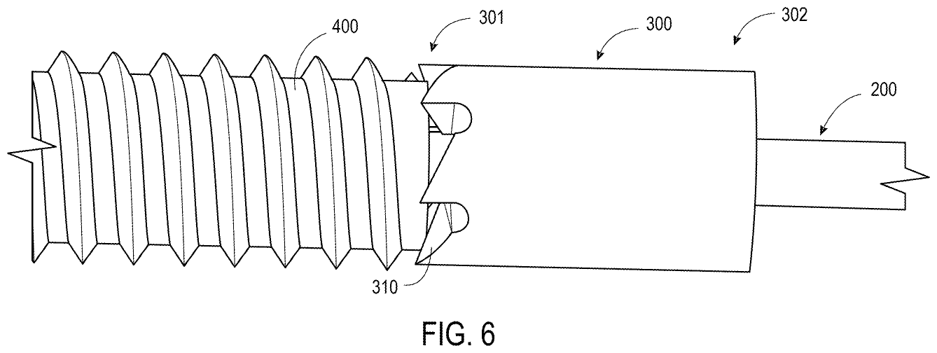

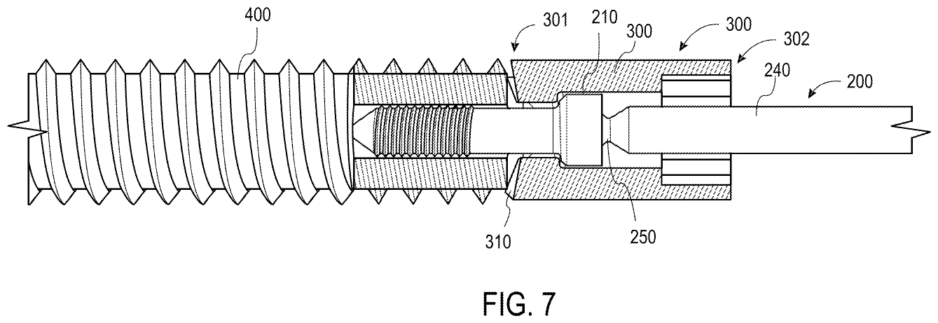

As a torque is applied to the elongated shaft 200 and the elongated shaft 200 advances into the cannulated screw 400, the stop head 210 may transmit an axial force on the step portion 330 of the collar 300, drawing the collar 300 closer to the exposed surface 410 of the cannulated screw 400. Referring to FIGS. 6 and 7, the method may include continuing to apply a torque to the elongated shaft 200 until the collar 300 engages with at least a portion of the cannulated screw 400. The collar 300 may be engaged with the cannulated screw when the protrusions 310 of the collar 300 are sufficiently seated into the cannulated screw 400.

In some examples, continuing to apply a torque to the elongated shaft 200 may include continuing to apply a torque to the elongated shaft 200 until the neck portion 250 experiences torsional failure and the torque delivery shaft 240 breaks apart from the stop head 210. This may allow a user to ensure that they have applied enough axial force from the collar 300 to the cannulated screw 400 so that the collar 300 and the cannulated screw 400 are secured together, but not too much force to further damage the cannulated screw 400. The use of the neck portion 250 may provide the surgeon with an objective means to ensure that enough axial force has been applied to the collar 300 to sufficiently mount to the cannulated screw 400 without having to go by feel or some other subjective measure.

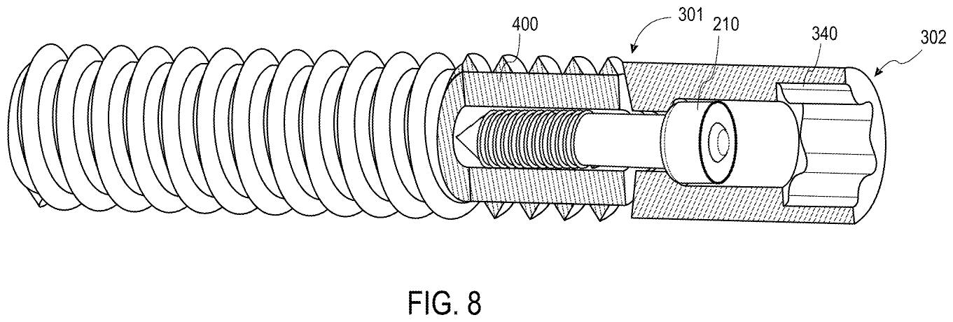

FIG. 8 illustrates the cannulated screw removal device 100 after the torque delivery shaft 240 has broken apart from the stop head 210. As can be seen in this figure, the breaking away of the torque delivery shaft 240 may expose the driver recess 340.

It is important to ensure the collar 300 is sufficiently mounted to the cannulated screw 400 before attempting to remove the cannulated screw 400. Specifically, if the collar 300 is not secured to the cannulated screw 400 and a torque is applied to the collar 300, the protrusions 310 of the collar 300 may slip and may become damaged. Additionally, the surface of the screw 400 may become damaged, which may make removal of the cannulated screw even more difficult.

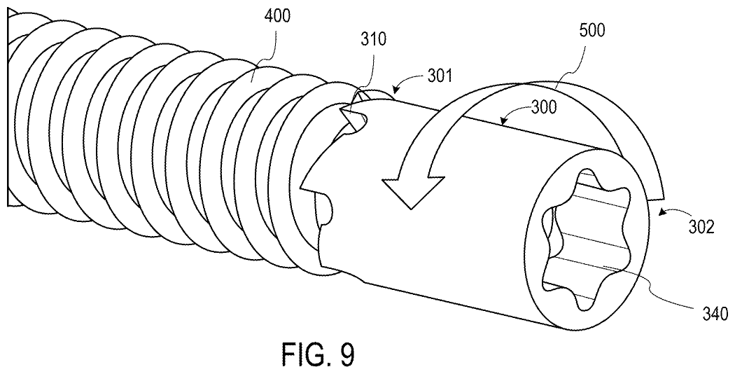

As illustrated in FIG. 9, after enough axial force (to mount the collar 300 to the screw 400, for example, by the torque delivery shaft 240) has been applied from the collar 300 to the cannulated screw 400, a torque 500 may be applied to the collar 300 to remove the screw 400. The torque 500 applied to the collar 300 may be transferred to the cannulated screw 400, such as through the protrusions 310 of the collar 300. In some examples, the torque 500 applied to the collar 300 may be a reverse torque, meaning the torque 500 is applied in the opposite direction of the threads of the cannulated screw 400. In some examples, the reverse torque may be to the left/counter-clockwise (when the threads of the cannulated screw are “right-handed”). In other examples, the reverse torque may be to the right/clockwise (when the threads of the cannulated screw are “left-handed”).

In some examples, the torque 500 may be applied by a driver, such as a manual or automatic driver. In this example, the method may include inserting the driver into the driver recess 340 before applying a torque 500. In some embodiments, the torque may be applied directly to the collar 300.

The method may include applying a torque to the collar 300 until the screw or screw fragment 400 is removed from the bone. In some examples, the method may include driving a new screw into the bone. The new screw may be one size larger than the original screw.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

### EMBODIMENTS

Various aspects of the subject matter described herein are set out in the following numbered embodiments:

Embodiment 1. A cannulated screw removal device comprising:
an elongated shaft comprising one or more threads configured to secure the elongated shaft to a cannula of a cannulated screw; and a collar comprising: a cannula extending from a distal end of the collar to a proximal end of the collar, wherein the cannula of the collar is configured to receive the elongated shaft; and one or more protrusions configured to engage with at least a portion of the cannulated screw, wherein the collar is configured to: surround at least a portion of the elongated shaft when the elongated shaft is received within the cannula of the collar; and deliver a torque to the portion of the cannulated screw through the one or more protrusions.

Embodiment 2. The cannulated screw removal device of embodiment 1, wherein the elongated shaft comprises: a stop head; and a cannulated screw engagement shaft configured to be inserted into the cannula of the cannulated screw, wherein a diameter of the stop head is larger than a diameter of the cannulated screw engagement shaft.

Embodiment 3. The cannulated screw removal device of any one of embodiments 1-2, wherein the cannula of the collar comprises: a first portion having a first diameter and configured to receive at least a portion of the cannulated screw engagement shaft; and a second portion having a second diameter and configured to receive the stop head, wherein the second diameter is greater than the first diameter.

Embodiment 4. The cannulated screw removal device of any one of embodiments 2-3, wherein the cannulated screw engagement shaft comprises the one or more threads.

Embodiment 5. The cannulated screw removal device of any one of embodiments 2-4, wherein the elongated shaft further comprises a torque delivery shaft connected to the stop head, wherein the stop head is disposed between the cannulated screw engagement shaft and the torque delivery shaft.

Embodiment 6. The cannulated screw removal device of embodiment 5, wherein the torque delivery shaft is removably coupled to the stop head

Embodiment 7. The cannulated screw removal device of any one of embodiments 5-6, wherein the elongated shaft further comprises a neck portion between the stop head and the torque delivery shaft, wherein the neck portion is configured to have a lower torsional strength than the cannulated screw engagement shaft, the stop head, and the torque delivery shaft such that the neck portion is configured to experience torsional failure at a lower torque force than the cannulated screw engagement shaft, the stop head, and the torque delivery shaft.

Embodiment 8. The cannulated screw removal device of any one of embodiments 1-7, wherein the one or more threads comprise left-handed threads.

Embodiment 9. The cannulated screw removal device of any one of embodiments 1-8, wherein the one or more threads comprise one or more cutting grooves.

Embodiment 10. The cannulated screw removal device of any one of embodiments 1-9, wherein the elongated shaft comprises a taper.

Embodiment 11. The cannulated screw removal device of any one of embodiments 1-10, wherein the one or more threads comprise a taper.

Embodiment 12. The cannulated screw removal device of any one of embodiments 1-11, wherein the cannula of the collar comprises a driver engagement recess configured to engage with a driver.

Embodiment 13. The cannulated screw removal device of any one of embodiments 1-12, wherein each of the one or more protrusions comprises a tip.

Embodiment 14. The cannulated screw removal device of embodiment 13, wherein the tip of each of the one or more protrusions is sharply pointed.

Embodiment 15. The cannulated screw removal device of any one of embodiments 13-14, wherein the tip of each of the one or more protrusions is on the left most side of the protrusion.

Embodiment 16. A method for removing a cannulated screw from bone using a cannulated screw removal device, the cannulated screw removal device comprising: an elongated shaft comprising one or more threads; and a collar configured to surround at least a portion of the elongated shaft, the method comprising: applying a torque to the elongated shaft in a first direction which causes the elongated shaft to screw into a cannula of the cannulated screw, securing the elongated shaft to the cannulated screw; continuing to apply a torque to the elongated shaft until the collar engages with at least a portion of the cannulated screw; and applying a torque to the collar in the first direction to remove the cannulated screw from the bone.

Embodiment 17. The method of embodiment 16, wherein the elongated shaft comprises a first portion comprising the one or more threads and a second portion connected to the first portion, wherein applying a torque to the elongated shaft comprises applying a torque to the second portion.

Embodiment 18. The method of any one of embodiments 16-17, wherein the elongated shaft further comprises a third portion between the first portion and the second portion, wherein the third portion is configured to have a lower torsional strength than the first portion and the second portion such that the third portion is configured to experience torsional failure at a lower torque force than the first and second portions, and wherein continuing to apply the torque comprises applying the torque to the elongated shaft until the third portion experiences the torsional failure causing the first portion and the second portion to separate.

Embodiment 19. A cannulated screw removal collar comprising: a cannula extending from a distal end of the cannulated screw removal collar to a proximal end of the cannulated screw removal collar, wherein the cannula of the cannulated screw removal collar is configured to receive an elongated shaft; and one or more protrusions configured to engage with at least a portion of a cannulated screw, wherein the one or more protrusions are disposed at a distal end portion of the collar, wherein the cannula of the cannulated screw removal collar comprises: a first portion having a first diameter and configured to receive at least a portion of the elongated shaft; and a second portion having a second diameter greater than the first diameter.

Embodiment 20. The cannulated screw removal collar of embodiment 19, wherein each of the one or more protrusions comprises a tip.

Embodiment 21. The cannulated screw removal collar of embodiment 20, wherein the tip of each of the one or more protrusions is sharply pointed.

Embodiment 22. The cannulated screw removal collar any one of embodiments 20-21, wherein the tip of each of the one or more protrusions is on the left most side of the protrusion.

Embodiment 23. The cannulated screw removal collar any one of embodiments 19-22, wherein the cannula of the cannulated screw removal collar comprises a driver engagement recess configured to engage with a driver.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the system of the present disclosure, there may be terms used like first, second, third, and fourth. These terms may be used for the purpose of differentiating one component from the other, but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A cannulated screw removal device comprising:
an elongated shaft comprising one or more threads configured to secure the elongated shaft to a cannula of a cannulated screw; and
a collar comprising:
a cannula extending from a distal end of the collar to a proximal end of the collar, wherein the cannula of the collar is configured to receive the elongated shaft; and
one or more protrusions configured to engage with at least a portion of the cannulated screw,
wherein the collar is configured to:
surround at least a portion of the elongated shaft when the elongated shaft is received within the cannula of the collar; and
deliver a torque to the portion of the cannulated screw through the one or more protrusions.

2. The cannulated screw removal device of claim 1, wherein the elongated shaft comprises:
a stop head; and
a cannulated screw engagement shaft configured to be inserted into the cannula of the cannulated screw, wherein a diameter of the stop head is larger than a diameter of the cannulated screw engagement shaft.

3. The cannulated screw removal device of claim 2, wherein the cannula of the collar comprises:
a first portion having a first diameter and configured to receive at least a portion of the cannulated screw engagement shaft; and
a second portion having a second diameter and configured to receive the stop head, wherein the second diameter is greater than the first diameter.

4. The cannulated screw removal device of claim 2, wherein the cannulated screw engagement shaft comprises the one or more threads.

5. The cannulated screw removal device of claim 2, wherein the elongated shaft further comprises a torque delivery shaft connected to the stop head, wherein the stop head is disposed between the cannulated screw engagement shaft and the torque delivery shaft.

6. The cannulated screw removal device of claim 5, wherein the torque delivery shaft is removably coupled to the stop head.

7. The cannulated screw removal device of claim 5, wherein the elongated shaft further comprises a neck portion between the stop head and the torque delivery shaft, wherein the neck portion is configured to have a lower torsional strength than the cannulated screw engagement shaft, the stop head, and the torque delivery shaft such that the neck portion is configured to experience torsional failure at a lower torque force than the cannulated screw engagement shaft, the stop head, and the torque delivery shaft.

8. The cannulated screw removal device of claim 1, wherein the one or more threads comprise left-handed threads.

9. The cannulated screw removal device of claim 1, wherein the one or more threads comprise one or more cutting grooves.

10. The cannulated screw removal device of claim 1, wherein each of the one or more protrusions comprises a tip.

11. The cannulated screw removal device of claim 10, wherein the tip of each of the one or more protrusions is sharply pointed.

12. The cannulated screw removal device of claim 10, wherein the tip of each of the one or more protrusions is on the left most side of the protrusion.

13. A method for removing a cannulated screw from bone using a cannulated screw removal device, the cannulated screw removal device comprising:
an elongated shaft comprising one or more threads; and
a collar configured to surround at least a portion of the elongated shaft,
the method comprising:
applying a torque to the elongated shaft in a first direction which causes the elongated shaft to screw into a cannula of the cannulated screw, securing the elongated shaft to the cannulated screw;
continuing to apply a torque to the elongated shaft until the collar engages with at least a portion of the cannulated screw; and
applying a torque to the collar in the first direction to remove the cannulated screw from the bone.

14. The method of claim 13, wherein the elongated shaft comprises a first portion comprising the one or more threads and a second portion connected to the first portion, wherein applying a torque to the elongated shaft comprises applying a torque to the second portion.

15. The method of claim 14, wherein the elongated shaft further comprises a third portion between the first portion and the second portion, wherein the third portion is configured to have a lower torsional strength than the first portion and the second portion such that the third portion is configured to experience torsional failure at a lower torque force than the first and second portions, and wherein continuing to apply the torque comprises applying the torque to the elongated shaft until the third portion experiences the torsional failure causing the first portion and the second portion to separate.

16. A cannulated screw removal collar comprising:
a cannula extending from a distal end of the cannulated screw removal collar to a proximal end of the cannulated screw removal collar, wherein the cannula of the cannulated screw removal collar is configured to receive an elongated shaft; and
one or more protrusions configured to engage with at least a portion of a cannulated screw, wherein the one or more protrusions are disposed at a distal end portion of the collar,
wherein the cannula of the cannulated screw removal collar comprises:
a first portion having a first diameter and configured to receive at least a portion of the elongated shaft; and
a second portion having a second diameter greater than the first diameter.

17. The cannulated screw removal collar of claim 16, wherein each of the one or more protrusions comprises a tip.

18. The cannulated screw removal collar of claim 17, wherein the tip of each of the one or more protrusions is sharply pointed.

19. The cannulated screw removal collar of claim 17, wherein the tip of each of the one or more protrusions is on the left most side of the protrusion.

20. The cannulated screw removal collar of claim 16, wherein the cannula of the cannulated screw removal collar comprises a driver engagement recess configured to engage with a driver.
